(19) United States
(12) Patent Application Publication
(10) Pub. No.: US 2010/0056564 A1
(43) Pub. Date: Mar. 4, 2010

(54) Association of an anti-atherothrombotic agent and an anti-platelet-aggregation agent

(75) Inventors: Laure Cloarec-Blanchard, Paris (FR); Stefano Corda, Paris (FR); Laurence Lerond, Marly-Le-Roi (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: LES LABORATOIRES SERVIER, SURESNES (FR)

(21) Appl. No.: 12/590,489

(22) Filed: Nov. 9, 2009

Related U.S. Application Data
(63) Continuation of application No. 10/574,119, filed on Mar. 30, 2006.

(30) Foreign Application Priority Data
Oct. 3, 2003 (FR) 03.11595

(51) Int. Cl. A61K 31/4365

(52) U.S. Cl. 514/301

(57) ABSTRACT

The present invention relates to a new association of an anti-atherothrombotic agent and an anti-platelet-aggregation agent.

The new invention relates to a new association of an anti-atherothrombotic agent and an anti-platelet-aggregation agent and to pharmaceutical compositions containing them.

More specifically, the present invention relates to the association of a specific TP receptor antagonist and clopidogrel.

Thromboxane A₂ (TXA₂) is an unstable metabolite of arachidonic acid which is involved in the pathogenesis of numerous cardiovascular illnesses. Thromboxane A₂ is a powerful platelet activator but is also a powerful vasoconstrictor which has cell proliferative and pro-adhesive properties.

TXA₂ and other metabolites of arachidonic acid such as endoperoxides (PGG₂-PGH₂), HETEs and isoprostanes exert their action by way of common receptors called TP receptors (thromboxane-prostaglandins-endoperoxides).

Numerous research studies have recently been carried out with the aim of preventing phenomena associated with the excessive production of thromboxane A₂ in the cardiovascular and neurovascular systems. Among such antagonists, those described in the Patent Specification EP 648 741 have been found to be powerful and selective antagonists of TP receptors, to be active via the oral route and to have a long duration of action.

More specifically, the compound (A) of formula (I):

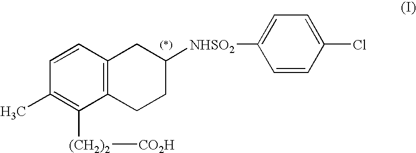

in racemic form or in the form of an optically pure isomer and also pharmaceutically acceptable salts thereof, has been found to be a powerful anti-atherothrombotic agent.

Compound A is a specific antagonist of TP receptors, more especially a specific antagonist of thromboxane A₂ and of prostaglandin-endoperoxide (PGG₂-PGH₂) receptors, imparting to that compound a powerful atherothrombotic effect.

In general, the formation of a thrombus after rupture of an atheroma plaque results from the interaction between the circulating platelets and the collagen of the basal lamina of the vascular endothelium exposed to the blood flow. This phenomenon is called atherothrombosis.

Collagen is present in the basal lamina of the vascular wall and is the determining factor for the thrombogenicity of atheromatous lesions in humans and in animals.

Platelet adhesion to the fibres of the collagen takes place via the collagen receptor and involves the adhesion of the platelets, their activation and their aggregation.

Platelet activation is accompanied by the liberation of two principal agonists, ADP and thromboxane A₂, which bind to their respective receptors (P2Y, TP) on the adjacent platelets and amplify the adhesion and platelet aggregation.

ADP is also present in the blood as a circulating mediator, while thromboxane A₂ is a powerful secondary mediator which is formed in the activated platelets from arachidonic acid via cyclo-oxygenase 1.

Thromboxane A₂ not only promotes thrombosis but also induces a dysfunction of the vascular wall (vasoconstriction) and promotes the proliferation and inflammatory infiltration of the wall.

Among the anti-platelet treatments currently available, aspirin allows the inhibition of platelet production from thromboxane A₂, while clopidogrel inhibits platelet aggregation induced by ADP.

ADP and thromboxane A₂ play an important and complementary role in the formation of the arterial thrombus.

Compound A acts by blocking platelet aggregation induced by thromboxane A₂ and the other TP receptor ligands, whatever their origin, platelet or extra-platelet.

It further acts by inhibiting vasoconstriction induced by thromboxane A₂ and by opposing endothelial dysfunction and the proliferation and inflammation of the vascular wall.

We have now found, in humans, that the association of compound A with clopidogrel allows, surprisingly, a synergy to be obtained in terms of anti-thrombotic activity.

In fact, because compound A and clopidogrel act on completely different pathways of platelet aggregation, it was especially advantageous to associate those two compounds in order to envisage a new therapeutic approach.

Surprisingly, it has been found that the association of compound A and clopidogrel allows substantial synergy to be obtained in terms of activity, which could not have been foreseen from any teaching of the literature. This association allowed an improvement in the anti-thrombotic effect evaluated by the inhibition of collagen-induced platelet aggregation ex vivo.

In the course of that test it was shown that the anti-thrombotic activity of compound A is potentiated in the presence of clopidogrel and increases in extremely substantial and entirely unforeseeable manner. Furthermore, that association has a good acceptability profile.

In the associations according to the invention, compound (A) and clopidogrel can be present in the form of pharmaceutically acceptable salts.

Among the addition salts of compound (A) there may be mentioned, without implying any limitation, addition salts with a pharmaceutically acceptable base, such as sodium, potassium, tert-butylamine and diethylamine salts etc.

Preference will be given to the use of the sodium salt.

Among the addition salts of clopidogrel, preference will be given to the hydrogen sulphate.

In the associations according to the invention, compound (A) preferably has the absolute configuration (R).

The present invention relates also to pharmaceutical compositions comprising an association of compound (A) and clopidogrel, where appropriate in the form of pharmaceutically acceptable salts, together with one or more appropriate inert, non-toxic excipients.

Among the pharmaceutical compositions according to the invention there may be mentioned more especially those that are suitable for oral, parenteral or nasal administration, tablets or dragées, sublingual tablets, gelatin capsules, suppositories, creams, ointments, dermal gels etc.

The dosage can be varied according to the nature and severity of the condition, the administration route and also the age and weight of the patient.

In the compositions according to the invention, the amounts of the active ingredients are in the range from 1 to 300 mg for compound (A) and from 10 to 600 mg for clopidogrel.

The compositions according to the invention are accordingly useful in the treatment of cardiovascular illnesses involving the activation of TP receptors and also in the treatment of the consequences of those illnesses. Those conditions include, without implying any limitation, acute coronary syndrome, stable or unstable angina, endothelial dysfunction, vascular illnesses associated with atherosclerosis, hypertension, diabetes and heart failure, and in the prevention and treatment of disorders of the vascular, cardiovascular or neurovascular system and of thrombo-embolic disorders associated especially with atherosclerosis, auricular fibrillation and invasive surgical procedures in cardiology, neurology, vascular pathology and radiology (angioplasty, installation of stents, bypasses, catheters etc.).

### Measurement of the Inhibition of Collagen-Induced Platelet Aggregation:

10 mg of compound A and 75 mg of clopidogrel were administered orally for three days to 18 volunteers previously treated with 75 mg of clopidogrel for 7 days. The effect of the association of compound A and clopidogrel was compared with the effects of compound A and clopidogrel administered separately.

In the course of the test, the percentage inhibition of platelet aggregation ex vivo induced by collagen (5 μg/ml) was calculated by measuring the platelet aggregation on citrated platelet-rich plasma (PRPc) with the aid of an aggregometer.

The results obtained are as follows:
administration of compound A on its own leads to 35% inhibition,
administration of clopidogrel on its own leads to 11% inhibition,
administration of the association of compound A and clopidogrel leads to 62% inhibition.

The results show very clearly that administration of those two compounds in association allows a synergy effect to be obtained in terms of collagen-induced platelet aggregation.

That anti-aggregation effect obtained by virtue of the association is accordingly superior to the sum of the effects of the two products taken separately. There is nothing in the literature to suggest that type of result.

The results suggest that the association may prove to be beneficial in acute or chronic conditions requiring an increased anti-thrombotic effect associated with a vascular effect (acute treatment or secondary prevention of neurovascular or cardiovascular illnesses).

1. A composition comprising a combination of compound (A) of formula (I): 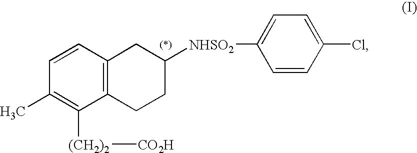
optionally in the form of an optical isomer, or a pharmaceutically acceptable salt thereof, and clopidogrel or a pharmaceutically acceptable salt thereof.

2. The composition of claim 1, wherein compound (A) has the (R) configuration.

3. The composition of claim 1, wherein compound (A) is in the form of a sodium salt.

4. The composition of claim 1, wherein clopidogrel is in the form of a hydrogen sulphate.

5. A pharmaceutical composition comprising as active ingredients a combination of compound (A) of formula (I): 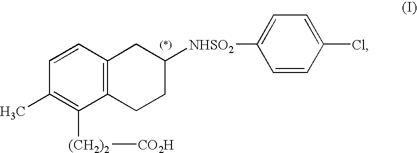
optionally in the form of an optical isomer, or a pharmaceutically acceptable salt thereof, and clopidogrel or a pharmaceutically acceptable salt thereof, in combination with one or more pharmaceutically acceptable, inert excipients or carriers.

6. The pharmaceutical composition of claim 5, wherein the compound (A) of formula (I) has the (R) configuration.

7. The pharmaceutical composition of claim 5, wherein compound (A) is in the form of a sodium salt.

8. The pharmaceutical composition of claim 5, wherein clopidogrel is in the form of a hydrogen sulphate.

9. The pharmaceutical composition of claim 5, wherein the amounts of active ingredients are in the respective ranges of from 1 to 300 mg for the compound (A) of formula (I) and from 10 to 600 mg for clopidogrel.

10. A method for treating a cardiovascular illness involving activation of TP receptors, such method comprising administering to a living animal body, including a human, a therapeutically effective amount of a composition of claim 1.

11. The method of claim 10, wherein the cardiovascular illness is selected from acute coronary syndrome, stable or unstable angina, endothelial dysfunction, and vascular illnesses.

12. A method for treating a disorder of the vascular, cardiovascular, or neurovascular system or a thrombo-embolic disorder, such method comprising administering to a living animal body, including a human, a therapeutically effective amount of a composition of claim 1.

13. The method of claim 12, wherein the thrombo-embolic disorder results from atherosclerosis, auricular fibrillation and invasive surgical procedures in cardiology, neurology, vascular pathology and/or radiology.
